(19) United States
(12) Patent Application Publication
(10) Pub. No.: US 2024/0360403 A1
(43) Pub. Date: Oct. 31, 2024

(54) TRANSGENE EXPRESSION OF LIGNINOLYTIC ENZYMES IN FUNGI

(71) Applicant: Battelle Memorial Institute, Richland, WA (US)

(72) Inventors: Ziyu Dai, Richland, WA (US); Ana L. Robles, Paso, WA (US); Joonhoon Kim, Bothell, WA (US); Scott E. Baker, Richland, WA (US); Jon K. Magnuson, Richland, WA (US); Beth A. Hofstad, Richland, WA (US); John M. Gladden, Martinez, CA (US)

(21) Appl. No.: 18/649,932

(22) Filed: Apr. 29, 2024

Related U.S. Application Data
(60) Provisional application No. 63/462,735, filed on Apr. 28, 2023.

(51) Int. Cl. C12N 1/14; C08H 7/00; C12N 9/02; C12N 9/08; C12N 15/80; C12R 1/645

(52) U.S. Cl. CPC C12N 1/14; C08H 6/00; C12N 9/0061; C12N 9/0065; C12N 15/80; C12N 2500/20; C12N 2501/998; C12R 2001/645; C12Y 111/01013; C12Y 111/01014; C12Y 111/01016

(57) ABSTRACT

Fungus with incorporated of heterologous ligninolytic enzyme to enhance effective conversion of lignocellulosic feedstocks to various chemical productions.

### PRIORITY

This application claims the benefit of priority of U.S. provisional patent application No. 63/462,735, filed on Apr. 28, 2023, the benefit of priority of which is claimed hereby, and which is incorporated by reference herein in its entirety.

### STATEMENT OF GOVERNMENT SUPPORT

This invention was made with Government support under Contract DE-AC0576RL01830 and DE-AC02-05CH11231 awarded by the U.S. Department of Energy. The Government has certain rights in the invention.

### BACKGROUND

Lignocellulosic materials are widely abundant sparking considerable interest in these materials for various biofuel applications. Lignocellulosic biomass such as wood waste, crop stalks and grasses are potentially sustainable sources of biomass for ethanol production. With most of the terrestrial biomass on earth being lignocellulosic, producing ethanol from lignocellulosic material has the potential to replace up to 30% of annual petroleum consumption in the United States while significantly reducing greenhouse gas emissions. Moreover, the use of lignocellulosic material in ethanol production does not encounter food production pressures as with other crop sources for ethanol such as corn.

The abundant sugars contained in lignocellulosic materials are blocked from traditional ethanol-producing fermentation reactions because they typically occur in a complex polymerization of lignin and celluloses that is difficult to hydrolyze into soluble sugars for fermentation. Since lignin is highly resistant to water penetration and enzymatic breakdown, it represents a significant barrier to isolating cellulose and other sugars for use in production of both paper and ethanol biofuel. Similarly, the high degree of polymerization of cellulose in woody biomass is also a significant barrier to efficient biofuel conversion and requires chemical and/or enzymatic hydrolysis to produce soluble sugars for fermentation.

Current methods using chemical and enzymatic processes for lignin removal and cellulose hydrolysis are cost prohibitive and inefficient to support industrial-scale lignocellulosic ethanol production.

### SUMMARY

One embodiment provides a modified filamentous fungus transformed with at least two polynucleotides encoding manganese peroxidase (mnP), versatile peroxidase, laccase or lignin peroxidase (lip). In one embodiment, the fungus is transformed with manganese peroxidase (mnP) and laccase. In another embodiment, the fungus is transformed with manganese peroxidase (mnP) and lip. In one embodiment, the fungus is transformed with manganese peroxidase (mnP), laccase, versatile peroxidase and lip. In one embodiment, the polynucleotide comprises at least one intron.

One embodiment further comprising a disruption in expression of one or more of transcriptional factor of protease (prtT), subunit of Goli mannoyltransferase complex (mnn9), and V-SNARE binding protein (vsm1) genes of the fungus. In one embodiment, expression of prtT gene is disrupted. In another embodiment, expression of prtT and vsm1 genes is disrupted. In one embodiment, the fungus is transformed with manganese peroxidase and the prtT gene is disrupted.

One embodiment provides a method of culturing the modified fugus in culture medium comprising a protein selected from hemoglobin, soy protein, skim milk protein, serum albumin or combination thereof. In one embodiment, the fungus is cultured at a pH of about 4 to about 5. In one embodiment, the fungus is cultured in the presence of manganese.

One embodiment provides a method to increase expression of ligninolytic enzyme from a filamentous fungus comprising transforming said fungus with at least two polynucleotides encoding manganese peroxidase (mnP), versatile peroxidase, laccase or lignin peroxidase (lip) and culturing said transformed fungus so as to express at least two of manganese peroxidase (mnP), versatile peroxidase, laccase or lignin peroxidase (lip). In one embodiment, the fungus is transformed with manganese peroxidase (mnP) and laccase polynucleotides. In one embodiment, the fungus is transformed with manganese peroxidase (mnP) and lip polynucleotides. In one embodiment, the fungus is transformed with manganese peroxidase (mnP), laccase, versatile peroxidase and lip polynucleotides. In another embodiment, the polynucleotide comprises at least one intron. One embodiment further provides a disruption in expression of one or more of transcriptional factor of protease (prtT), subunit of Goli mannoyltransferase complex (mnn9), and V-SNARE binding protein (vsm1) genes. In one embodiment, expression of prtT gene is disrupted. In one embodiment, the expression of prtT and vsm1 genes is disrupted. In one embodiment, the fungus is transformed with manganese peroxidase and the prtT gene is disrupted. One embodiment further comprises culturing said transformed fungus in culture medium comprising a protein selected from hemoglobin, soy protein, skim milk protein, serum albumin or combination thereof. In one embodiment, the fungus is cultured at a pH of about 4 to about 5. In one embodiment, manganese is added to the culture medium.

One embodiment further comprises harvesting one or more of manganese peroxidase (mnP), versatile peroxidase, laccase or lignin peroxidase (lip).

One embodiment provides a method to degrade lignin comprising contacting said lignin with the modified fungus described herein or one or more enzymes harvested from said fungus.

### BRIEF DESCRIPTION OF THE FIGURES

Embodiments of the invention are described below with reference to the following accompanying drawings.

FIG. 1 illustrates an embodiment of transgene expression constructs.

FIG. 2 depicts GFP expression in the mnp2-gfp fusion expression.

FIGS. 3A-3B provide a comparison of flask and test tube culture with or without 5 g/l of bovine hemoglobin 200 rpm.

FIG. 4 illustrates the effect of different hemoglobin concentrations on mnP production (30 Degrees Celsius/200 rpm/2 days).

FIGS. 5A-5C illustrate the effect of bovine hemoglobin on cellular metabolism.

FIGS. 6A-6B illustrate the effects of shaking speed on mnP2 production.

FIGS. 7A-7C illustrate the effects of culture conditions on mnP production and its stability.

FIGS. 8A-8B illustrate a comparative evaluation of transgene expression of mnp1, mnp2, mnp3, and mnp5.

FIGS. 9A-9B illustrate the effects of selected genes involved in protein secretory pathway on A5IL97 and mnP production. Key for FIG. 9A: ATCC11414: citric acid production strain of A. niger; A5IL97: thermotolerant, IL-tolerant beta-glucosidase; pyrG: recyclable auxotrophic (uracil/uridine) marker; prtT: global regulator of secreted peptidase production; mnn9: subunit of Golgi mannosyltransferase complex; vsm1: V-snare binding protein; late secretory pathway; deletion increases secretion in Sc. Δvsm1 and Δmnn9 show improved expression of heterologous enzyme vs. control. Dual deletion of prtT and vsm1 shows an unexpected synergist 65% improvement over prtT alone. Key for FIG. 9B: Pc-mnp2: 1 of 5 manganese peroxidase genes from Phanerochaete chrysosporium: Codon optimized cDNA of Pc mnp2 (4172-2, 4173-7); Codon optimized cDNA+first intron of Pc mnp2 (4176-4); Genomic DNA of Pc mnp2 in 11414: prtT (4174-11 4175-8); Genomic DNA of Pc mnp2 in 11414 (4175-4); Genomic DNA of Pc mnp3 (4181-7), &Pc-mnp5 (4198-4) (7 introns in mnp2)

FIGS. 10A-10B illustrate transgene expression of vp2 gene and the effects of bovine hemoglobin on its production.

FIGS. 11A-11B illustrates transgene expression of lac26 gene under the control of ubi1 or gla1 promoter.

FIG. 12 illustrates the effects of bovine hemoglobin on Lac26 production.

FIG. 13 illustrates mnP decoloring selected dyes (percentage decoloring/Time in hours).

FIG. 14 illustrates the lac26 decoloring selected dyes (percentage of dye degradation/time course in hours).

FIG. 15 demonstrates the effects of different gene disruptions on A5IL97 production in A. niger. (ATCC11414, A5IL97, A5IL97/pyrGΔ, A5IL97/prtTΔ, A5IL97/mnn9Δ, A5IL97/vsm1Δ and A5IL97/vsm1Δ/prtTΔ)

FIGS. 16A-16B depict the catalytic cycle of MnP (Hofrichter, 2002) and manganese peroxidase uses in biotechnological applications.

FIG. 17 illustrates the effects of bovine hemoglobin on mnP2 production (30° C./200 rpm/2 days).

FIGS. 18A-18B illustrate the effects of shaking on mnP2 production.

FIGS. 19A-19B illustrate the effects of initial pH in the culture medium on mnP2 production (A) and the effects of culture temperature on mnP2 production at pH 5.5 (B).

FIG. 20 depict spore aging effects.

FIG. 21 depicts effects of antioxidants, such as L-glutathione/L-ascorbic acid, on Mnp2 production (antioxidant effect on Mnp2 activity (ABTS); cultured in modified A. niger minimum medium (30° C./200 rpm).

FIG. 22 depicts effects of culture medium and different preparations of hemoglobin.

FIGS. 23A-23B illustrate a comparative evaluation of transgene expression of Smnp2, Sgla1, cmnP2, gmnP2a, gmnP2b, gmnP3, pmnP5, CGlmnP1 and cPomnP4.

FIG. 24 depicts VP2 activity in transgenic strains.

FIG. 25 illustrates the effects of hemin and hemoglobin on Mnp2 activity. Heme alone was not stimulatory, but intact hemoglobin has an effect.

FIG. 26 demonstrates the combined expression of lignin deconstruction enzymes Mnp2 and Lac26. No Mn or malonate added to stimulate manganese peroxidase activity, therefore indicative of laccase activity.

FIG. 27 provides protein alignments of 4 isoforms of mnP genes identified in P. chrysosporium. (SEQ ID NOS: 1-4)

FIG. 28 illustrates the effects of additional proteins (bovine hemoglobin, soy protein, skim milk protein, and bovine serum albumin) on manganese peroxidase (mnP) production in transgenic A. niger strain. The proteins were prepared by either autoclave or 0.2 μm filtration. The results show that the proteins, such as bovine hemoglobin, soy protein, skim milk protein, and bovine serum albumin have augmented the mnP production. For the mnP production, the bovine hemoglobin is the most effective and bovine serum albumin is the least effective.

FIG. 29 depicts the effects of additional manganese on manganese peroxidase (mnP) production. The results demonstrate show that additional 5 mg/L of manganese into the cultures led to the highest mnP production.

FIG. 30 shows the effects of additional ion on manganese peroxidase (mnP) production. The results demonstrate that additional 5 mg/L of ion into the cultures led to the highest mnP production.

FIG. 31 demonstrate the effects of additional ion on manganese peroxidase production in A. nigers with disruption of prtT or double disruption of both prtT and vsm1 genes leads to substantial improvement in the mnP production.

### DETAILED DESCRIPTION

The following description describes embodiments of the present disclosure. It will be clear from this description of the disclosure that the disclosure is not limited to these illustrated embodiments but that the disclosure also includes a variety of modifications and embodiments thereto. Therefore, the present description should be seen as illustrative and not limiting. While the disclosure is susceptible of various modifications and alternative constructions, it should be understood, that there is no intention to limit the disclosure to the specific form disclosed, but, on the contrary, the disclosure is to cover all modifications, alternative constructions, and equivalents falling within the spirit and scope of the disclosure.

In one aspect, Aspergillus niger is an industrial filamentous fungal strain for global citric acid production, which is being explored herein for other value-added chemical production, as well as incorporation of heterologous ligninolytic enzyme production to enhance the effective conversion of lignocellulosic feedstocks to various chemical productions. Protein production and secretion in various microbes, including the filamentous fungi, is governed by both biotic and abiotic factors. Thus far, over a dozen proteins were identified, which can significantly influence the protein production and secretion in different eukaryotic microbes. Abiotic factors such as, oxygen, temperature, pH, hemoglobin and antioxidants can influence the overall protein production.

Provided herein is the transgene expression of ligninolytic enzymes, such as manganese peroxidase, laccase, versatile peroxidase, in a fungal organism, such as industrial filamentous fungal Aspergillus niger. Embodiments describe modulating protein secretory paths, reduction of protease degradation, increase of the copy number of the transgene expression with different isoforms, and culture conditions.

Further embodiments describing the effects of the prtT (transcriptional factor of proteases), mnn9 (subunit of Goli mannoyltransferase complex), laccase and vsm1 (V SNARE binding protein) on glucosidase (A5IL97, an ionic liquid tolerant thermophilic cellulase) production in Aspergillus niger via gene disruption are provided. When individual genes were disrupted, the A5IL97 activity was about 2.6 to 9.2 times higher than the transgenic parent strain (A5IL97). When vsm1 was further deleted from the A5IL97/prtT transgenic strain, the A5IL97 activity was increased 14.9-fold as compared to the transgenic parent strain. This strategy is also applicable to the transgene expression of other related proteins in, for example, A. niger.

Transgene expression of manganese peroxidase (mnP) was examined and modified in A. niger, for example. Provided herein is the evaluation of the transgene expression of all four isoforms of mnP (pcmnP1, pcmnP2, pcmnP3, and pcmnP5) from P. chrysosporium, glmnP1 from Ganoderma lucidum, pomnP4 from Pleurotus ostreatus. It was found that pcmnP2 had the highest transgene expression by the enzyme activities. When the prtT gene was disrupted in the pcmnP2 transgenic strain, the mnP2 activity was increased by 2.2-fold. Various factors such as, nitrogen, oxygen, temperature, pH, hemoglobin, antioxidants, and spore age all exert their effects on mnP production in A. niger. Further improvement of the mnP production can occur with disruption of other related genes, such as vsm1 or mnn9 or overexpression of other pcmnP isoforms in the pcmnP2/prtT transgenic strain background. The overall conditions for mnP production were established in A. niger.

Described herein is the effect of prtT disruption in combination of disruption of VSM1 and/or other genes, such as mnn9, to improve A5IL97 or manganese peroxidase production.

Various advantages and novel features of the present disclosure are described herein and will become further readily apparent to those skilled in this art from the following detailed description. In the preceding and following descriptions embodiments of the disclosure have been shown and described by way of illustration of the best mode contemplated for carrying out the invention. As will be realized, embodiment described herein are capable of modification in various respects without departing from the disclosure. Accordingly, the drawings and description of embodiments set forth hereafter are to be regarded as illustrative in nature, and not as restrictive.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, pro-peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

The term “expression” includes any step involved in the production of a polypeptide including, but not limited to, transcription, post-transcriptional modification, translation, post-translational modification, and secretion.

The term “expression vector” means a linear or circular DNA molecule that comprises a polynucleotide encoding a polypeptide and is operably linked to control sequences that provide for its expression.

The term “operably linked” means a configuration in which a control sequence is placed at an appropriate position relative to the coding sequence of a polynucleotide such that the control sequence directs expression of the coding sequence.

The term “host cell” means a fungus that is susceptible to transformation, transfection, transduction, or the like with a nucleic acid construct or expression vector comprising a polynucleotide for use in the present invention. The term “host cell” encompasses any progeny of a parent cell that is not identical to the parent cell due to mutations that occur during replication.

The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment-Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

$$( Identical Deoxyribonucleotides \times 100 ) / ( Length of Alignment - Total Number of Gaps in Alignment )$$

The term “variant” means a polypeptide having activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

### Lignin/Lignocellulosic Material

Any desired lignocellulosic material can be used. In some embodiments, a lignocellulosic material comprises one or more types of wood. In some embodiments, for example, wood comprises one or more types of hardwood, softwood or mixtures thereof. In some embodiments, wood comprises one or more types of genetically modified woods or plants. In some embodiments, a lignocellulosic material comprises plant leaves and/or stalks including, but not limited to, corn stover. Moreover, in some embodiments, lignocellulosic material comprises one or more grasses including, but not limited to, switchgrass. Lignocellulosic material suitable for use in methods described herein, in some embodiments can be obtained as waste products from various applications such as timber harvesting and associated processing, agricultural harvesting and associated processing and/or landscape clearing and maintenance applications.

In some embodiments, a lignocellulosic material suitable for use in one or more methods described herein has a moisture content of at least about 10%. In some embodiments, a lignocellulosic material has a moisture content of at least about 15% or at least about 20%. A lignocellulosic material, in some embodiments, has a moisture content of at least about 30% or at least about 50%. In some embodiments, a lignocellulosic material has a moisture content ranging from about 10% to about 80% or from about 20% to about 60%. In some embodiments, moisture can be added to the lignocellulosic material prior to a treatment described herein.

In some embodiments, a lignocellulosic material is provided in particulate form.

In some embodiments, for example, wood is provided in particulate form for administering a method described herein. In some embodiments, wood and/or other forms of lignocellulosic material can be chipped or ground into particulate form in preparation for administering a method described herein.

### Fungal Types

Modified fungi are used in the methods provided herein for degrading lignin of the lignocellulosic material. In some embodiments, the fungus is operable to degrade lignin and cellulose of the lignocellulosic material. In some embodiments, for example, a lignin degrading fungus comprises one or more white rot fungi.

In some embodiments, the fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

In some embodiments of methods described herein, a modified lignin degrading fungus is applied to the lignocellulosic material. Lignin degrading fungus or fungi can be applied to the lignocellulosic material for any desired period of time. In some embodiments, for example, a lignin degrading fungus is applied to the lignocellulosic material for a time period sufficient to degrade a desired amount of lignin.

The time period over which a lignin degrading fungus is applied to a lignocellulosic material can be dependent on several factors including the identity of the fungus, identity of the lignocellulosic material, amount of the lignocellulosic material and/or the surrounding environmental conditions. In some embodiments, for example, a lignin degrading fungus is applied to the lignocellulosic material for a time period of at least about hours, days, or 1 week. In some embodiments, a lignin degrading fungus is applied to the lignocellulosic material for a time period of at least about 2 weeks or at least about 3 weeks. In some embodiments, a lignin degrading fungus is applied to the lignocellulosic material for a time period ranging from about 1 week to about 5 weeks. In some embodiments, a lignin degrading fungus is applied to the lignocellulosic material for a time period ranging from about 2 weeks to about 4 weeks. In some embodiments, a lignin degrading fungus is applied to the lignocellulosic material for a time period less than about 1 week.

In some embodiments, a lignin degrading fungus or fungi is operable to degrade at least about 1 weight percent of the lignin content of the lignocellulosic material. In some embodiments, a lignin degrading fungus or fungi degrades at least about 5 weight percent or at least about 10 weight percent of the lignin content of the lignocellulosic material. In some embodiments, a lignin degrading fungus or fungi degrades at least about 25 weight percent or at least about 50 weight percent of the lignin content of the lignocellulosic material. In some embodiments, a lignin degrading fungus or fungi degrades from about 1 weight percent to about 90 weight percent of the lignin content of the lignocellulosic material. In some embodiments, a lignin degrading fungus or fungi degrades from about 5 weight percent to about 75 weight percent of the lignin content of the lignocellulosic material. In some embodiments, a lignin degrading fungus or fungi degrades from about 10 weight percent to 60 weight percent of the lignin content of the lignocellulosic material.

In some embodiments, a lignin degrading fungus is applied to a lignocellulosic material in any suitable manner known to one of skill in the art. In some embodiments, for example, a lignin degrading fungus is applied to a lignocellulosic material by liquid phase techniques. In some embodiments, a lignin degrading fungus is applied to a lignocellulosic material by contacting the lignocellulosic material with a lignocellulosic material containing the lignin degrading fungus.

In some embodiments, a container of a bioreactor described herein has a volume sufficient to hold at least 1 ton of lignocellulosic material. In some embodiments, a bioreactor container has a volume sufficient to at least 2 tons or at least 3 tons of lignocellulosic material. In some embodiments, a bioreactor container has a volume sufficient to at least 4 tons or at least 5 tons of lignocellulosic material. In some embodiments, apparatus for introducing at least one lignin degrading fungus to the lignocellulosic material comprises a fluid transport system with one or more injection points for liquid phase application of the at least one lignin degrading fungus.

In some embodiments, a bioreactor described herein further comprises one or more agitators for mixing or mechanically agitating the lignocellulosic material in the container. Additionally, in some embodiments, a bioreactor described herein comprises apparatus suitable for autoclaving or otherwise sterilizing the lignocellulosic material in the container, such as apparatus for steam treating the lignocellulosic material.

In some embodiments, a bioreactor described herein further comprises climate control apparatus for regulating the temperature and moisture content of the lignocellulosic material and/or surrounding environment. In some embodiments, a bioreactor comprises water spraying apparatus to control the moisture content of the lignocellulosic material as well as heating and cooling apparatus for controlling temperature of the lignocellulosic material and surrounding environment.

### Enzymes

Some embodiments provide genetically modified strains of fungus for improved protein production and secretion.

In some embodiments, exogenous manganese peroxidase (AA2/hydrogen-peroxide oxidoreductase/EC1.11.1.13), versatile peroxidase (AA2/hydrogen-peroxide oxidoreductase/EC1.11.1.16), lignin peroxidase (LiP) (AA2/(3,4-Dimethoxyphenyl) methanol or hydrogenperoxide oxidoreductase/EC1.11.1.14) and/or laccase (AA1_1/Benzenediol oxygen oxidoreductase or 4 Benzenediol/EC1.10.3.2) is expressed in a fungus (creating transgenic organisms), such as A. niger. In other embodiments, a prtT, vsm1, and or mnn9 genes are mutated so as to be non-functional (or deleted) from the fungi. In some embodiments, the fungi comprise at least two of: exogenous manganese peroxidase expression, exogenous versatile peroxidase expression, exogenous laccase expression, disruption in prtT gene, disruption in vsm1 gene, and/or disruption in mnn9 gene.

In another aspect, the isolated polypeptides having activity are encoded by SEQ ID NO: 1, 2, 3 or 4 (FIG. 27) or at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity thereto.

In another aspect, the isolated polypeptides having activity are variants of SEQ ID NOS: 1-4 comprising a substitution, deletion, and/or insertion at one or more (e.g., several) positions. In an embodiment, the number of amino acid substitutions, deletions and/or insertions introduced into the polypeptide are up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physicochemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant mutant molecules are tested for cellulolytic enhancing activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127) and CRISPR/Cas.

CRISPR/Cas and/or mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

### Expression

Polynucleotides encoding polypeptides having activity can be isolated and utilized to practice the methods of the present invention, as described herein.

The techniques used to isolate or clone a polynucleotide encoding a polypeptide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

A polynucleotide encoding a polypeptide having activity may be operably linked to one or more control sequences that direct the expression of the coding sequence in a suitable host cell under conditions compatible with the control sequences.

The polynucleotide may be manipulated in a variety of ways to provide for expression of the polypeptide (such a codon optimization). Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Terminators for filamentous fungal host cells can be obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

### Expression Vectors

A polynucleotide encoding a polypeptide and various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression. The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector can contain one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Fungal cells may be transformed by a process involving protoplast formation, transformation of the protoplasts, and regeneration of the cell wall. Suitable procedures for transformation of Aspergillus and Trichoderma host cells are described in EP 238023, Yelton et al., 1984, Proc. Natl. Acad. Sci. USA 81:1470-1474, and Christensen et al., 1988, Bio/Technology 6:1419-1422. Suitable methods for transforming Fusarium species are described by Malardier et al., 1989, Gene 78:147-156, and WO 96/00787.

### Culture/Abiotic Factors

The host cells are cultivated in a nutrient medium suitable for growth using methods known in the art. For example, the cells may be cultivated by shake flask cultivation, or small-scale or large-scale fermentation (including continuous, batch, fed-batch, or solid state fermentations) in laboratory or industrial fermenters in a suitable medium. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

In some embodiments, the pH, temperature, shaking speed are taken into account.

Bioprocess development showed improved production dramatically by adding hemoglobin or other proteins. In some embodiments, one or more exogenous proteins are added to the nutrient medium, including, but not limited to, hemoglobin (such as bovine hemoglobin), soy protein, skim milk protein, and serum albumin (such as bovine serum albumin).

Embodiments of compositions and methods described herein are further illustrated by the following non-limiting example.

### Example

### Example I—Transgene Expression of Ligninolytic Enzymes Manganese Peroxidase, Versatile Peroxidase, and Laccase in Industrial Filamentous Fungal Aspergillus niger

Aspergillus niger is for global citrate production, which is being explored for other chemical production. Incorporation of production of related ligninolytic enzymes into this host enhances the effective conversion of lignocelluloses to various chemical productions.

Protein production and secretion in various microbes, is governed by both biotic and abiotic factors. Thus far, over a dozen proteins, which influence protein production and secretion in different eukaryotic microbes have been identified. Via gene disruption, the effects of prtT (transcriptional factor of proteases), mnn9 (subunit of Goli mannoyltransferase complex), and vsm1 (V-SNARE binding protein) on beta-glucosidase (A5IL97, an ionic liquid-tolerant thermophilic cellulase) production in A. niger was studied. When the individual gene candidates affecting secretion were disrupted, the A5IL97 activity was about 2.6 to 9.2 times higher than the parent strain (A5IL97). When the vsm 1 was deleted in A5IL97/prtTΔ transgenic strain, the A5IL97 activity was increased 14.9 folds in comparison to the A5IL97 strain.

Transgenic expression of manganese peroxidase (mnP) and hybrid versatile peroxidase (VP2.0) were further examined in A. niger. Transgene expression of all isoforms of mnP (pcmnP1, pcmnP2, pcmnP3, and pcmnP5) from Phanerochaete chrysosporium, glmnP1 from Ganoderma lucidum, pomnP4 from Pleurotus ostreatus, and a synthetic versatile peroxidase (VP2.0) were evaluated. It was found that pcmnP2 had the highest expression as assayed by enzyme activity. When prtT gene was disrupted in the pcmnP2 transgenic strain, the mnP2 activity was increased by 2.2-fold. Various factors, such as nitrogen, oxygen, temperature, pH, hemoglobin, antioxidants, and spore age all exert effects on mnP production in A. niger. The positive effect of addition of hemoglobin on the VP2.0 was also observed. Further improvement of the mnP production can bed brought about by disruption of additional genes including vsm 1 and mnn9, such by CRISPR/Cas9-based genome editing or over-expression of other pcmnP isoforms in the pcmnP2/prtTΔ transgenic strain background.

### INTRODUCTION

Lignocellulose is the major polysaccharide component of global plant mass, which consists of hemicellulose, lignin and cellulose. Lignin is a complex aromatic heteropolymer comprising phenylpropanoid ary-C3 units linked via a variety of ether and carbon-carbon linkages. Lignin is an energy-dense, heterogenous polymer comprising 10-03% of lignocellulosic biomass, which is closely intertwined with the cellulose and hemicellulose in plant cell walls and is the second most abundant biopolymer on Earth after cellulose. The breakdown of plant lignocellulose to glucose monomers is the basis for second-generation cellulosic bio-ethanol production, but the lignin polymer is highly resistant to breakdown. There is therefore considerable interest in methods to depolymerize lignin. Current methods for cellulosic bio-ethanol production use a DMR (deacetylation and mechanical refining) process (Chen et al., 2016), which liberates high monomeric sugar yields for biotransformation.

Large quantities of lignin are produced from paper and pulping industry, and lignocellulosic biorefineries. Currently, these industries leave approximately 140 million tons of lignin per year that were simply burned for heat and power supply (Kristensen et al., 2009; Lau and Dale, 2009; Lau et al., 2010; Zhou et al., 2014), which places a low value on lignin. However, research is focused on renewable fuels, chemicals, and polymers production from polymeric sugars of lignocellulosic biomass. In contrast, the research on lignin conversion into valuable bioproducts is still limited. Processes to convert lignin to valuable bioproducts could accommodate extra costs for recovery of lignin by capital intensive pretreatments (Schell et al., 2003).

Lignin like other lignocellulosic components should be depolymerized prior use in various industrial applications. Two common approaches are currently used for lignin depolymerization: thermochemical depolymerization and biological depolymerization. Various microorganisms, mainly fungi and bacteria, are capable of degrading lignin with the help of lignin modification enzymes (Ahmad et al., 2010). Earlier studies on the microbial degradation of lignin have primarily focused on breakdown by white-rot and brown-rot fungi, which are able to mineralize lignin and release CO₂ (Hatakka, 1994; Kirk and Farrell, 1987). White-rot fungi have been explored for a supplementary means for co-remediation of heavy metal and organic pollutants (Chen et al., 2022; Reddy, 1995), which produce a range of extracellular ligninolytic enzymes, including manganese peroxidases (MnPs; E.C.1.11.1.13), lignin peroxidases (LiPs; E.C.1.11.1.14), and versatile peroxidases (E.C.1.11.1.16), and laccases (E.C.1.10.3.2). However, different white rot fungi produce a different set of ligninolytic enzymes, such as Phanerochaete chrysosporium mainly produces both manganese peroxidase and lignin peroxidase (Vanden Wymelenberg et al., 2009), while Pleurotus eryngii is known for production of versatile peroxidase (Camarero et al., 2000) and Trametes sp. C30 for the laccase (Klonowska et al., 2002).

Despite the study of fungal lignin degradation since the discovery of ligninolytic enzymes in the mid-1980s (Glenn et al., 1983; Hatakka, 1994; Pointing, 2001; Tien and Kirk, 1983), there is as yet no commercial enzyme mixture for biocatalytic processes to depolymerize lignin, in part due to the practical challenges of protein production via natural or genetic engineering. The natural decomposition of lignin by abiotic and biotic processes releases sustainable amounts of CO₂ into the atmosphere. Converting lignin components into value-added products there are promising pathways to upgrade lignin and further contribute to decarbonization. Therefore, a consortium system will be an effective approach to de-polymerize and convert lignin into selected products. Ligninolytic enzymes, manganese peroxidase and lignin peroxidase from P. chrysosporium, laccase from Trametes sp. C30, and versatile peroxidase from P. eryngii were examined herein-their transgene expression and production in industrial filamentous fungus A. niger. Materials and Methods

### Strains and Media

The Escherichia coli strain Top10 was used for routine plasmid DNA preparation. A. pseudoterreus (ATCC 32359) and A. niger (ATCC 11414) from the American Type Culture Collection (Rockville, MD, USA), were grown on complete medium (CM) or potato dextrose agar (PDA) plates at 30° C. for culture maintenance and spore preparation. About 1×10⁴ to 1×10⁵ spores were inoculated on CM agar (petri dish) plates and incubated for four days at 30° C. Spores were harvested by washing with 5 to 10 ml sterile 0.4% Tween 80 (polyoxyethylenesorbitan monooleate) and pelleted by centrifugation at 2500 g for 5 min. The spores were re-suspended in sterile 0.4% Tween 80 and enumerated with a hemocytometer. Aliquots of the resulting spore suspension (about 10⁸˜10⁹ spores/ml) were used to inoculate different agar-plates or liquid cultures. The preparation of PDA, CM and minimal medium (MM) followed the description of Bennett and Lasure (Bennett and Lasure, 1991). All strains used are shown in Table 1.

TABLE 1
Strains

| Strain | Genotype | Reference |
|---|---|---|
| St-4129 | prtTΔ::ble, gla1p:cmnp1:gpdAt, hph | This work |
| St-4172 | prtTΔ::ble, gla1p:cmnp2:gpdAt, hph | This work |
| St-4173 | prtTΔ::ble, gla1p:Sgla1:cmnp2:gpdAt, hph | This work |
| St-4174 | prtTΔ::ble, gla1p:gmnp2a:gpdAt, hph | This work |
| St-4175 | prtTΔ::ble, gla1p:gmnp2b:gpdAt, hph | This work |
| St-4176 | prtTΔ::ble, gla1p:orig-cmnp2:gpdAt, hph | This work |
| St-4180 | prtTΔ::ble, gla1p:gmnp2b-gfp:gpdAt, hph | This work |
| St-4181 | prtTΔ::ble, gla1p:gmnp3:gpdAt, hph | This work |
| St-4194 | prtTΔ::ble, gla1p:cvp2:gpdAt, hph | This work |
| St-4198 | prtTΔ::ble, gla1p:gmnp5:gpdAt, hph | This work |
| St-4206 | prtTΔ::ble, ubi1p:clac26:gpdAt, hph | This work |
| St-4207 | prtTΔ::ble, gla1p:clac26:gpdAt, hph | This work |

Preparation of Transgene Expression Constructs for Gene Over-Expression or Gene Disruption in A. niger.

The β-alanine pathway transgene expression cassette was described in detail by Pomraning et al., 2021. The β-alanine pathway transgene expression cassette with the hph marker gene was prepared with the same set of oligo pairs except those used for hph marker gene. The β-alanine pathway transgene expression cassette with additional aspartate aminotransferase gene (Apaat1, jgi|Aspte1|7965|ATET_04402) was prepared by oligo pairs of AptefF/AptefR and ApaatF/Apaat, which was used for random integration of A. pseudoterreus. The PCR fragments for aspartate aminotransferase gene (aat1, GenBank: EHA22111) over-expression construct (ble, teflP:aat:pgk1T) were bleF1/bleR1 for the bacterial bleomycin resistance gene (ble), Ptef1F/Ptef1R for translation elongation factor 1 (tef1) promoter of A. niger, aatF/aatR for the aat1 cDNA of A. niger, and TpgkF1/TpgkR1 for the transcriptional terminator of phosphoglerate kinase (pgk, jgi/aspin7/1147902) gene of A. niger. All PCR fragments were assembled together with pBSK(−) linearized by HindIII and PstI restriction enzymes with Gibson assembly master mix (NEB, Ipswich, MA, USA). Similarly, the over-expression transgene construct for pyruvate carboxylase were prepared with the oligo pairs of bleF1/bleR2 (ble), TpgkF2/TpgkR1 (Tpgk), pycF/pycR for A. niger pyruvate carboxylase cDNA (pyc; jgi/Aspni7/1031996), and Pmbf1F/Pmf1R for the A. niger mbf1 gene (jgi/Aspni7/1145066) promoter or their combination, where pyc overexpression cassette was incorporated into the downstream of aat1 overexpression cassette right after the TpgkF2/TpgkR1 (Tpgk) fragment. The over-expression of A. niger monocarboxylate transporter (mct1, jgi/Aspni7/1163060), the oligo pairs of nat1F/nat1R for nourseothricin N-acetyl transferase (nat) of Streptomyces noursei, Pmbf1/Pmbf1R for A. niger mbf1 gene promoter and mctF/mctR for A. niger mct gene coding sequence were used for DNA fragment isolations. Finally, for the over-expression of A. niger uga2 (jgi/Aspni7/57046), the oligo pairs of Pubi4F/Pubi4R for A. niger ubi4 gene promoter, Anuga2F/Anuga2R for A. niger uga2 gene, PmdhF/PmdhR for A. niger malate dehydrogenase (mdh) gene promoter, and nptIIF/nptIIR for neomycin phosphotransferase II (nptII) gene were used to DNA fragment isolation.

Gene disruption constructs for ald6a (jgi/Aspni7/1182225), ald6b (jgi/Aspni7/201822), ald3 (jgi/Aspni7/1126238), uga2 (jgi/Aspni7/57046), and ochA (jgi/Aspni7/1145269) were prepared with the following oligo pairs: 5ald6aF/5ald6haR, hphF1/hphR1, and 3ald6haF/3ald6haR for ald6a gene; 5ald6bF/5ald6bR, hphF2/hphR2, and 3ald6bF/3ald6bR for ald6b gene; 5ald3F/5ald3R, hphF3/hphR3, and 3ald3F/3ald3R for ald3 gene; 5uga2F/uga2R, hphF5/hphR5, and 3uga2F/3uga2R for uga2; 5oahAF/oahAR, hphF4/hphR4, and 3oahAF/3oahAR for ochA gene.

For further genetic modification, the Tet-On/Cre-loxP system (Jiang et al., 2016) was incorporated into the transgenic strain. The loxP fragments were first introduced into the nptII marker gene with the oligo pair loxPnpt2F/loxPnpt2R and plasmid DNA of construct as a template. To construct a new b-alanine 3HP pathway transgene expression cassette with loxP-nptII as a selection marker, the plasmid DNA of the initial transgene expression construct used for A. pseudoterreus was used for the DNA template for isolation of panD, bapat, hpdh, elf3t and trpCt with the following oligo pairs: 2728pu4F/2729pu4R for A. niger ubi4 gene promoter, 2730panF/2731panR for panD gene and a part of elf3t, 2732bapatF/2733bapatR for bapat gene and a part of elf3t, 2734pus1F/2735pus1R for A. niger ubiS gene promoter, 2736pmbF/273pmbR for A. niger mbfA promoter, and 2738hpdhF/2739hpdhR for hpdh gene and A. nidulans trpC transcriptional terminator. All fragments were assembled into the plasmid DNA of the transgene expression construct linearized with XhoI restriction enzyme.

### Culture Methods

Pyrex 125 ml or 250 ml glass Erlenmeyer flasks were prepared by filling with 5% Contrad 70 (Decon Labs, Inc. King of Prussia, PA, USA) and soaked overnight to remove any potential residues on the inside surface of flasks prior to general dishwashing. Silicon sponge closures were used for all flask cultures. The biomass of transgenic clones and parent strain for genomic DNA isolation were prepared from 2 mL stationary CM cultures with proper antibiotics and grown in 13×100 mm glass culture-tubes for 24 to 36 hrs at 30° C. The biomass formed on the surface of the liquid culture medium was collected, frozen immediately in liquid nitrogen and dried in the VirTis benchtop manifold freeze dryer (SP Scientific, Gardiner, NY, USA). For 3-HP production, 35 ml of citric acid production (CAP) medium was prepared by following previous descriptions (Dai et al., 2004). Production medium B (RDM) (Riscaldati et al., 2000) or modified production medium B (mRDM) (Riscaldati et al., 2000) that contains 20×TE (trace elements: 4 mg/l CuSO₄·5H₂O; 110 mg/l FeSO₄·7H₂O; 14 mg/l MnCl₂·4H₂O; and 26 mg/l ZnSO₄·7H₂O) was also used. Deacetylated and disk refined corn stover that was enzymatically hydrolyzed (DDR-EH; Batch Jan. 19, 2005, 20190829) (https://doi.org/10.1186/s13068-015-0358-0) was kindly provided by the National Renewable Energy Laboratory.

Chemical-Mediated Protoplast Transformation of A. niger

The protoplast preparation and chemical-mediated transformation followed the method described by Dai et al. (2013) for A. niger. Briefly, the 14.4 kb plasmid DNA of 3-HP pathway transgene expression construct was linearized by restriction enzyme EcoRV and concentrated down to about 1 mg/ml with Microcon-30 kDa centrifugal filter unit (MilliporeSigma, Burlington, MA, USA). Ten microliters of the linearized plasmid DNA were used for protoplast transformation in A. niger. For transgene over-expression of A. niger aat1, pyc, the aat1-pyc, mct1, or uga2 gene in A. niger, about 3 to 5 mg of linearized plasmid DNAs by proper restriction enzymes were used for protoplast transformation. For the gene deletion construct of ald6a, ald6b, oah1, or uga2 gene homolog, about 1 mg of linearized plasmid DNAs by restriction enzyme Pmel was used for protoplast transformation in A. niger. Usually, about 5 to 12 transformed clones were picked randomly for evaluation of 3-HP production and the effects of selected genes on 3-HP production.

### Total Genomic DNA Isolation for POR, Southern Blotting Analysis, and Short-Read Whole Genomic DNA Sequencing.

Total genomic DNA was isolated from A. niger or A. pseudoterreus cells using a cetyltrimethylammonium bromide (CTAB) extraction method with some modifications. Briefly, 50 to 120 mg of lyophilized biomass and two 3.5 mm diameter glass beads were transferred into a 2 mL polypropylene micro-vial, where biomass was pulverized into fine power with a Mini-Beadbeater-8 (Bio Spec Products Inc., Bartlesville, OK, USA) for 50 seconds. The disrupted cells in microcentrifuge tubes were re-suspended with 800 to 900 μl of CTAB solution and incubated at 60° C. for 30˜45 min and inverted occasionally. The genomic DNA in the supernatant of the cell extract was extracted with 300 ml of phenol/chloroform solution and precipitated with 1 volume of 2-propanol. The genomic DNA was resuspended with 200 ml of 50TE (50 mM Tris-HCl, pH8.0 and 10 mM EDTA, pH8.0) and 25 μg of RNase and incubated for 30˜45 min at 50° C. After RNase treatment, the genomic DNA was extracted twice with 125 ml of phenol/chloroform solution and once with chloroform. The genomic DNA in the supernatants was precipitated with 1 M NaCl and 2 volume of 95% ethanol for 15 min at room temperature and centrifugation at 10,000×g for 8 min. Finally, the genomic DNA pellet was washed with 70% ethanol and was resuspended in 10 mM Tris-HCl (pH 8.0) buffer at 50° C. for 15 to 20 min and the concentration was determined with a Qubit fluorometer (Thermo Fisher Scientific, Waltham, MA, USA). Fifty to seventy ng of total genomic DNA were used for PCR analyses.

For Southern blotting analyses of heterologous expression of β-alanine pathway in either A. niger or A. pseudoterreus, one microgram of total genomics DNA was digested with the restriction endonuclease BamHI, EcoRV, or HindIII. The genomic DNA fragments were separated in 1% agarose gel electrophoretically and transferred onto the Hybond-N+ nylon membrane (GE Healthcare Bio-Sciences, Pittsburgh, PA, USA) with alkaline capillary transfer method. The 1.0 kb 3′-end of genomic DNA fragments of A. pseudoterreus cad1 gene was used for preparation of the biotin-labeled probe. The genomic DNA in the Hybond-N+ nylon membrane was hybridized with the biotin-labeled probe overnight at 60° C. in the Problot Hybridization Oven (Labnet International, Edison, NJ, USA). The genomic DNA on the hybridized membrane was visualized with North2South chemiluminescent detection kit (Pierce Protein Research Products, Rockford, IL, USA) in Analytikjena UVP ChemStudio (Analytik Jena US, Upland, CA, USA).

The short-read whole genomic DNA sequencing was carried by Azenta Life Sciences (South Plainfield, NJ, USA). The integration copy number was estimated by fold-increase of reads mapped to the expression construct versus background single copy regions of the genome. The sequenced short-reads were mapped to the reference genome sequence of A. niger ATCC 1015 (mycocosm.jgi.doe.gov/Aspni7/Aspni7.home.html) augmented with the overexpressed gene sequence using BWA-MEM (Li, 2013). The mapped reads were sorted using SAMtools (Li et al., 2009) and duplicate reads were marked using Picard Toolkit (github.com/broadinstitute/picard#citing) to produce BAM files for copy number estimation. The copy numbers of β-alanine 3HP pathway genes and engineered native genes were estimated using CNVnator (Abyzov et al., 2011). The mapped reads were counted using bin sizes of 100, 200, and 1,000 bp, and the read depth signal was partitioned into segments for each bin size. The average and standard deviation of read depth signal were evaluated for bin sizes of 100 and 200 bp, and copy number genotype was estimated based on the normalized read depth using the bin size of 100 bp.

### Metabolites Analysis by HPLC

The extracellular metabolites were quantified by HPLC. Twenty-five microliters of the samples filtered with 0.2 mm syringe filters were analyzed for 45 min using an Aminex HPX-87H ion exclusion column with a 1 mM H₂SO₄ flow of 0.6 ml/ml. The temperature of the column was 60° C. The refractive index at 45° C. and the UV absorption at 210 nm were measured.

### Sample Preparation for Metabolomics and Proteomics Analyses

Briefly, the culture supernatants or biomass (cell pellet) for A. niger or A. pseudoterreus were harvested at day 4. For quantification of extracellular metabolites diluted spent medium samples (by a ⅛ factor) were dried, prepared and analyzed as described previously (Pomraning et al, 2021). The cell pellets were extracted using the MPLex protocol (Nakayasu et al., 2016) and extracts were analyzed using GC-MS as explained previously in detail (Kim et al., 2015). The protein interlayer pellet was digested and prepared for global proteomics analysis and targeted proteomics analysis, the latter using heavy labeled peptides. Instrument acquisition and data analysis was done as described in a previous manuscript (Pomraning et al, 2021). Global proteomics data were generated using a Q Exactive Plus mass spectrometer (Thermo Fisher Scientific) in data-dependent acquisition mode.

### Results

Access of Manganese Peroxidase (mnP2) of P. chrysosporium Expression in A. niger

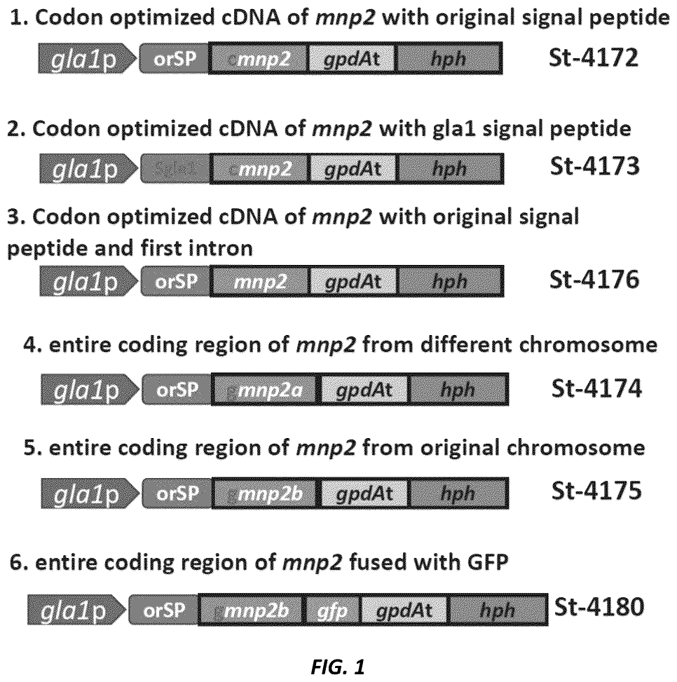

Although the mnp2 transgene expression has be demonstrated in transgenic A. niger and maize seed with its mature cDNA previously (Clough et al., 2006; Conesa et al., 2000), provided herein is the transgene expression of this gene re-evaluated in the citric acid production strain of A. niger ATCC 11414. The cDNA of mnp2 was de novo synthesized with A. niger codon optimization or original cDNA of P. chrysosporium (access No: jgi|Phchr4_2|6120750). The coding sequence of mnp2 genomic DNA was isolated from P. chrysosporium genomic DNA. The transgene expression constructs st-4172, st-4173, and st-4176 contain the synthetic cDNA under the control of A. niger glucoamylase (gla1p) promoter and transcriptional terminator of A. niger gpdA (gpdAt) gene (FIG. 1). The construct st-4172 contains the entire cDNA of mnP2 with A. niger codon optimization, while the construct st-4173 is the same of st-4172 except its secretory signal peptide (first 24 amino acids) that was replaced with the gla1 secretory signal peptide (first 21 amino acids, Sgla1). The construct st-4176 was original mnp2 cDNA with its first intron and under the control of the same promoter (gla1p) and transcriptional terminator (gpdAt). Similarly, the construct st-4174 and st-4175 were assembled with the entire coding region of mnp2 genomic DNA isolated by PCR with dikaryotic P. chrysosporium Burdsall ATCC24725 (Covert et al., 1992). Two allelic variants of mnp2 genes were isolated and confirmed by DNA sequencing. The st-4175 contains the mnp2 coding sequence that was identical to that reported in the literature. The mnp2-gfp fusion construct st-4180 was also prepared with gfp fused to the C-terminus of mnp2.

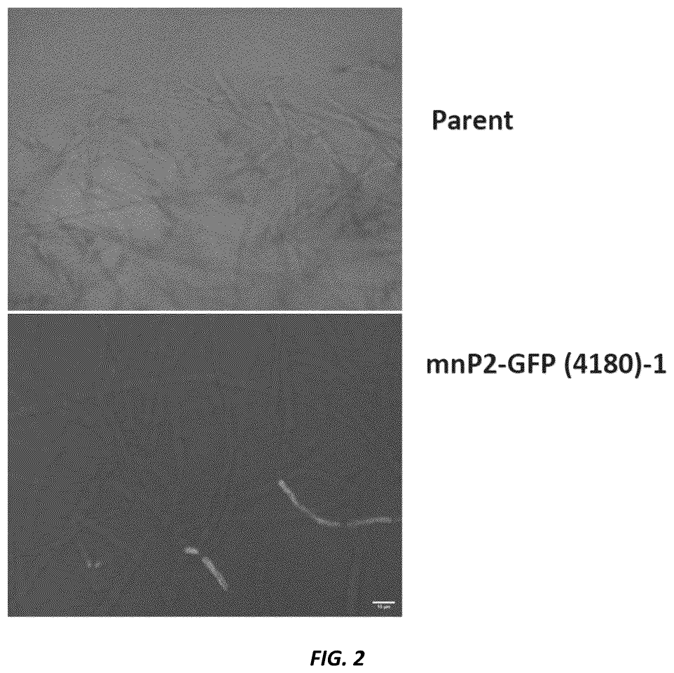

The transgenic strains of A. niger were generated with all five transgenic expression constructs. Manganese peroxidase activities were detected by spectrophotometry with the substrate of 2,2′-azino-bis (3-ethylbenothiazoline-6-sulfonic acid) (ABTS) in all transgenic strains grown in the 5 ml test tube cultures, especially, the transgenic strain 4175-8 having the highest activity (data not shown). The gfp in the transgenic strain st-4180 was visualized by confocal microscopy (FIG. 2).

The Effects of Bovine Hemoglobin on mnP Production in Transgenic A. niger st-4175-8 Strain

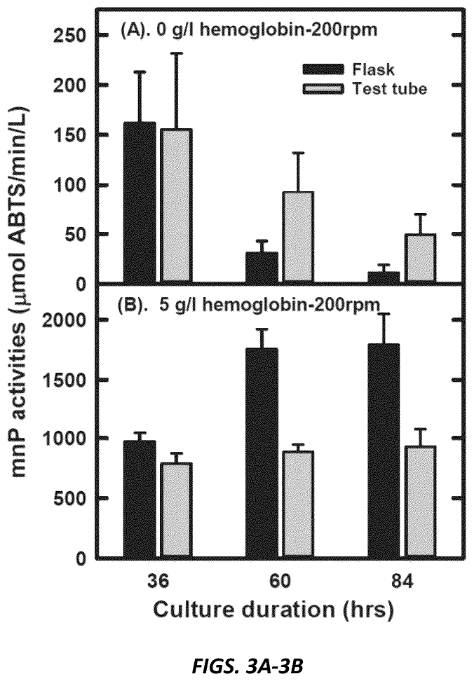

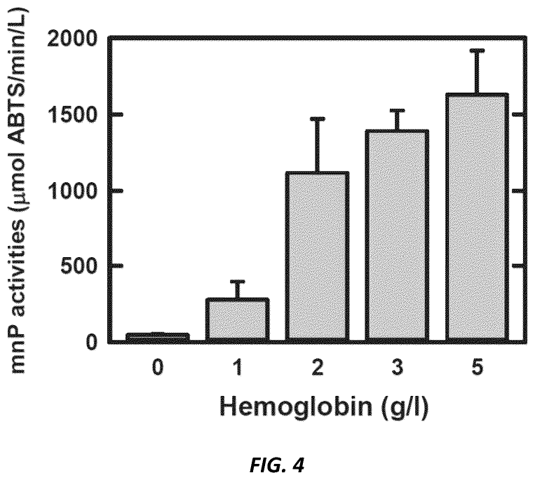

Previous study demonstrated that bovine hemoglobin positive role in mnP production in transgenic A. niger (Conesa et al., 2000). Provided herein is the study of the transgenic strain st-4175-8 for further evaluation of mnP production at different culture conditions. When the transgenic A. niger strain was grown with the modified minimum medium (mMM) in either shake flask or test tube, the mnP activity was about 155 mmol ABTS/min/L (U/L) at 36 hrs and sustainably decreased thereafter at both types of cultures, especially the shake flask cultures (FIG. 3A), suggesting the potential O₂ effects on mnP production in the cultures. In contrast, overall mnP activities in the mMM culture medium with 5 g/L of bovine hemoglobin (bHb) at 36 hrs were about 972 U/L in shake flask cultures and 785 U/L in test tube cultures, more than 5 times higher than that at regular mMM and reached 1753 U/L in shake flask cultures after 60 hrs growth, while 885 U/L in test tube cultures. The effects of different bHb concentrations on mnP production were examined as well (FIG. 4). At 0, 1, 2, 3, and 5 g/L bHb culture the mnP activities ranges from 48, 278, 1119, 1391, and 1629 U/L, respectively. About 2 g/L bHb is the minimal concentration to maintain high mnP activity in the cultures.

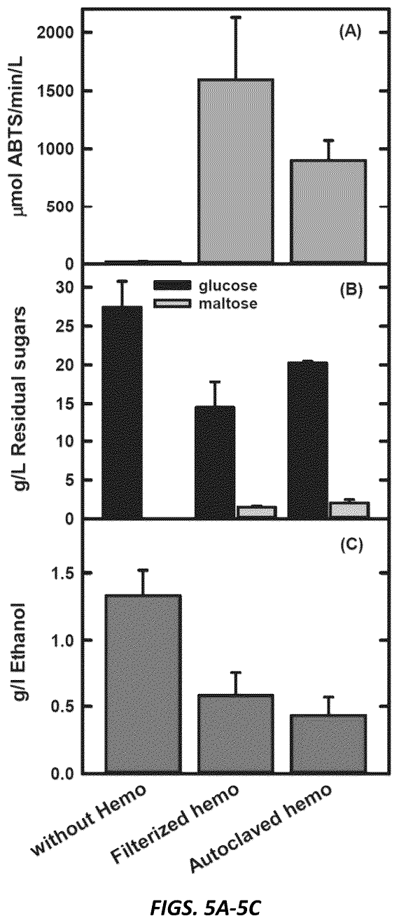

The effects of hemin on mnP production were also examined. About 0, 70, 250, 500, and 1000 mg/L hemin were added into the mMM culture medium and grown at 30° C. for 2 days. No improvement in mnP activities were detected in those cultures (data not shown), suggesting that hemin is not a component in bHb affecting mnP production. The residual sugars and ethanol accumulation in the culture supernatants were quantified by HPLC and shown in FIG. 5. Maltose in all cultures was effectively converted to glucose. About 54% of sugars were consumed in the cultures without bHb, while 73 or 63% of sugars were used in the cultures with bHb sterilized b filter-filtration or autoclave (FIG. 5B). Interestingly, the culture with bHb accumulated less ethanol compared to culture without bHb (FIG. 5C).

The Effects of pH, and Temperatures, Shaker Speed on mnP Production in Transgenic A. niger St-4175-8 Strain

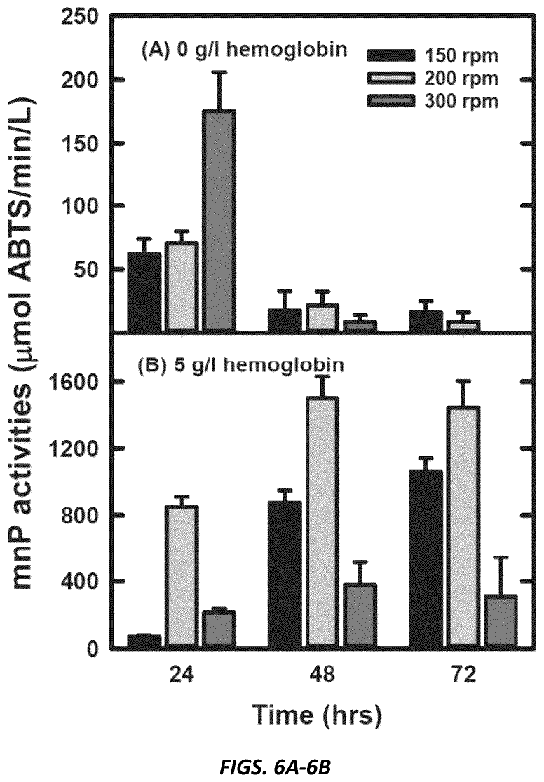

The effects of shaker speed, pH, and temperature on mnP production in transgenic strain st-4175-8 were quantified. The effects of shaker speeds on mnP production in the shaker flask cultures with or without 5 g/L bHb were determined at 24, 48, and 72 hrs. FIG. 6A shows that the mnP activities in the cultures without bHb after 24 hrs growth were 62, 70, and 175 U/L with the shaker speed of 150, 200, and 300 rpm, respectively. Thereafter, the mnP activities were dramatically decreased at the same culture conditions. The results demonstrated that 300 rpm is appears to be optimal in the same culture conditions without bHb.

When the 5 g/l bHb was added into the culture medium, the mnP activities in the cultures with 150 rpm were 73, 875, and 1059 U/L after 24, 48, and 72 hrs growth (FIG. 6B). At 200 rpm cultures, the mnP activities were 850, 1500, and 1450 U/L, while were 215, 377, and 309 U/L at 300 rpm cultures after 24, 48, and 72 hrs growth. The 200 rpm shaker speed was optimal to produce the highest mnP production in the shaker flask cultures with 5 g/L bHb.

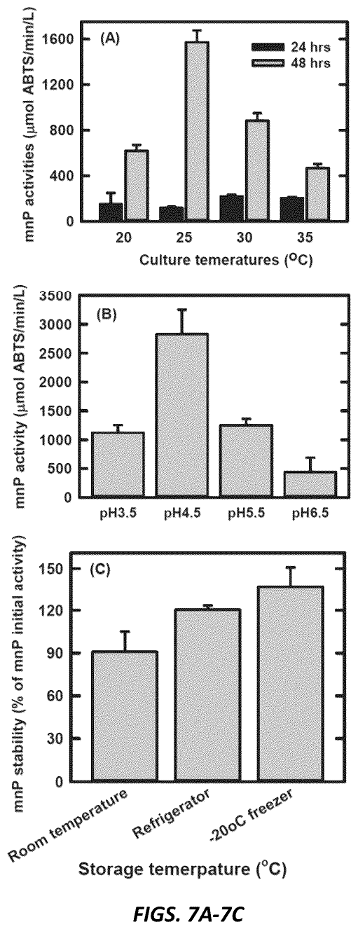

The effects of culture temperatures in the mMM with 5 g/L bHb were estimated after 24 and 48 hrs growth (FIG. 7A). The mnP activities after 24 hrs growth were 148, 112, 218, and 202 U/L and were 618, 1572, 885, 468 U/L after 48 hrs growth at 20, 25, 30, and 35° C. The effects of initial culture medium pH on mnP production in the transgenic strain st-4175-8 were determined in mMM with 5 g/L bHb (FIG. 7B). the mnP activities at pH 3.5, 4.5, 5.5, and 6.5 were 1122, 2826, 1250, and 436 U/L, respectively. The optimal pH in the initial culture medium with bHb was 4.5. The stability of mnP proteins were evaluated in the culture supernatants stored at room temperature, 4° C. refrigerator, and −20° C. freezer for 90 hrs. The results in FIG. 7C show that mnP only reduced 10% of its at room temperature and remains fully active at 4° C. or −20° C.

Comparative Examination of all Four Isoforms of P. chrysosporium mnP Expressed in A. niger.

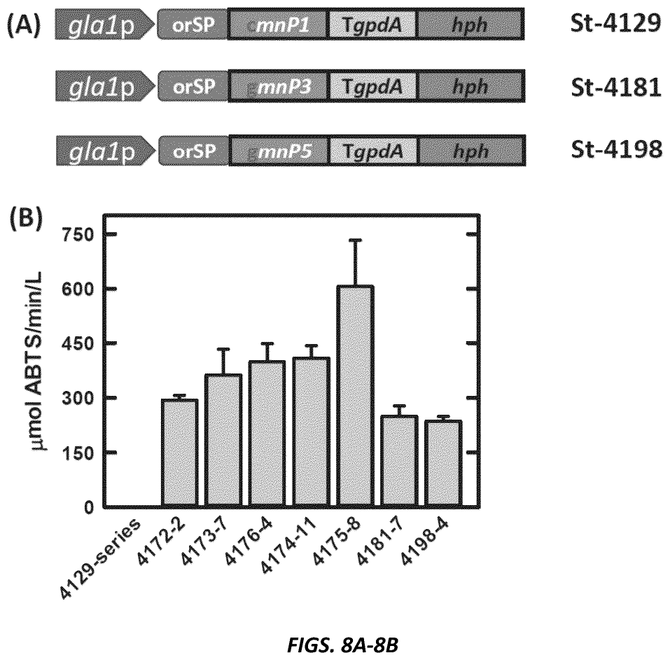

Transgene expressions of the other three isoforms of mnPs of P. chrysosporium were also examined in A. niger. The cDNA of mnp1 (cmnp1) was de novo synthesized with A. niger codon optimization. The coding regions of genomic DNAs for mnp3 (gmnp3) and mnp5 (gmnp5) were isolated by PCR with P. chrysosporium genomic DNA as templates. The transgene expressions of cmnp1, gmnp3, and gmnp5 under the control of A. niger gla1 (gla1p) promoter and transcriptional terminator of A. niger gpdA (gpdAt) gene were prepared (FIG. 8A). All three transgene expression cassettes were integrated into A. niger prtTD strain to generate transgenic strain st-4129, st-4181, and st-4186.

The mnP activities were evaluated at the same culture time and conditions in all transgenic strains (st-4129, st-4172, st-4273, st-4274, st-4275, st-4276, st-4281, & st-4298). The results in FIG. 8B show that mnp2, mnp3, and mnp5 except mnp1 were functionally expressed in the transgenic A. niger strains. The transgenic strain st-4175-8 has the highest mnP activity among the eight transgenic strains. The results also demonstrated that the genomic DNA from P. chrysosporium can be directly used for transgene expression in A. niger.

### Improvement of Overall mnP Production Via Modification of Protein Secretory Pathway

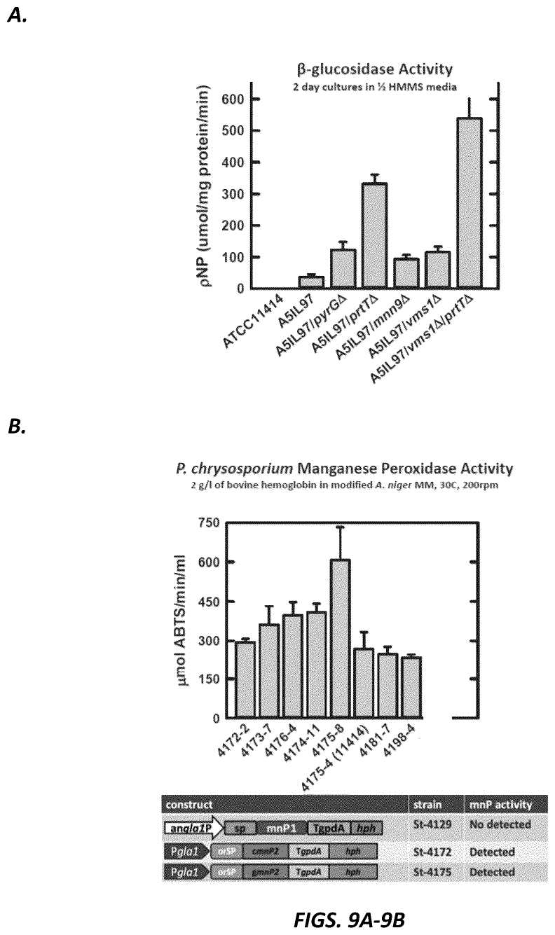

The transcriptional activator of protease (prtT), v-SNARE-interacting protein (vsm1), and mannosyltransferase (mnn9) all have negative effects on overall protein secretion. The transgenic A. niger strain containing the transgene expression of ionic liquid-tolerant thermophilic b-glucosidase (A5IL97) under the control of A. niger gla1 promoter and A. nidulans trpC transcriptional terminator, was selected to examine the effects of prtT, vsm1, and mnn9 on its production. Disruption of prtT, vsm1, or mnn9 gene in the transgenic strain had 9, 3, or 2.5 fold improvements in A5IL97 production (FIG. 9A). When prtT gene was disrupted in the A5IL97/vsm/D transgenic strain, the A5IL97 production had about 15 folds improvements. The similar approach was applied to mnP production in A. niger. The mnP activities were determined in the transgenic strains containing the same transgene expression construct st-4175 with either parent or prtTD strain background. The results in FIG. 9B show that disruption of prtT led to more than 2-fold improvement in mnP production.

Transgene Expression of Hybrid Laccase (Lac26) of Trametes sp. C30 and Plant-Fungal Hybrid Versatile Peroxidase (VP2.0) in Transgenic A. niger

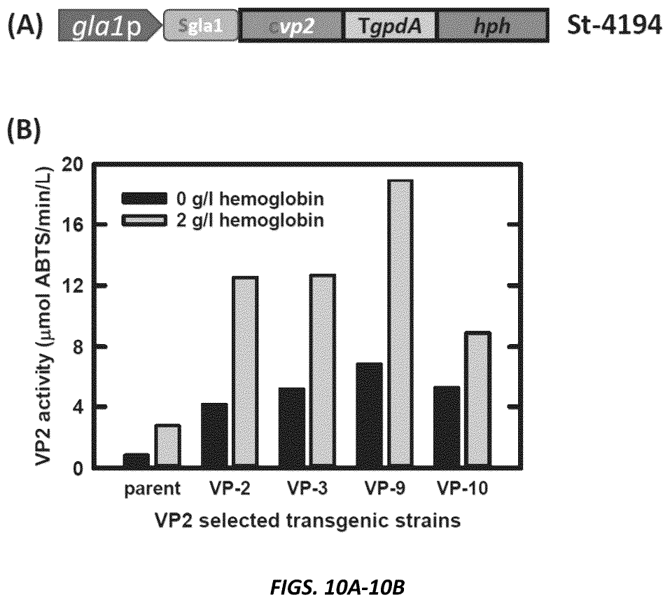

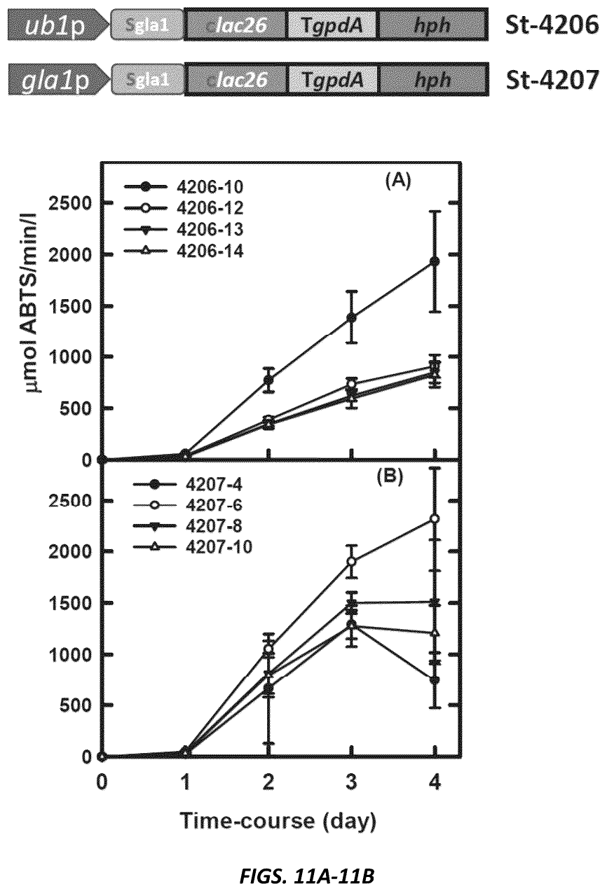

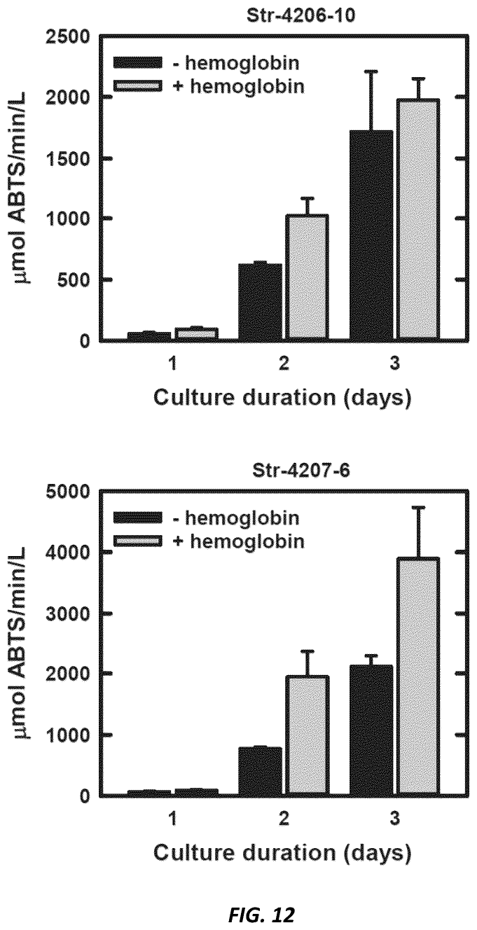

Transgene expression cassette for vp2 gene under the control of A. niger gla1 promoter and A. niger gpdA transcriptional terminator were constructed and integrated into 11414prtTD strain (FIG. 10A). The vp2 activities in different transgenic strains were determined with ABTS as a substrate for their assays and shown in FIG. 10B. The versatile peroxidase (VP) was functionally expressed in the transgenic A. niger strains and additional bHb enhanced the VP production. Transgene expression of lac26 under the control of A. niger ubi1 or gla1 promoter and A. niger gpdA transcriptional terminator (FIG. 11A). The lac26 activities were measured in the selected transgenic strains and shown in FIGS. 11B & 11C. The results indicated that lac26 expression was slightly higher under the control of gla1 promoter (the highest lac26 production strain: st-4207-6, 2315 U/L) than that under the control of ubi1 promoter (the highest lac26 production strain: st-4206-10, 1932 U/L). When 2 g/L bHb was added into the culture medium for the strain st-4206-10 and st-4207-6 growth, the VP activities were much higher in the medium with bHb than that without additions of bHb into the culture medium (FIG. 12).

### Evaluation of mnP and Lac26 Decoloring Selected Dyes

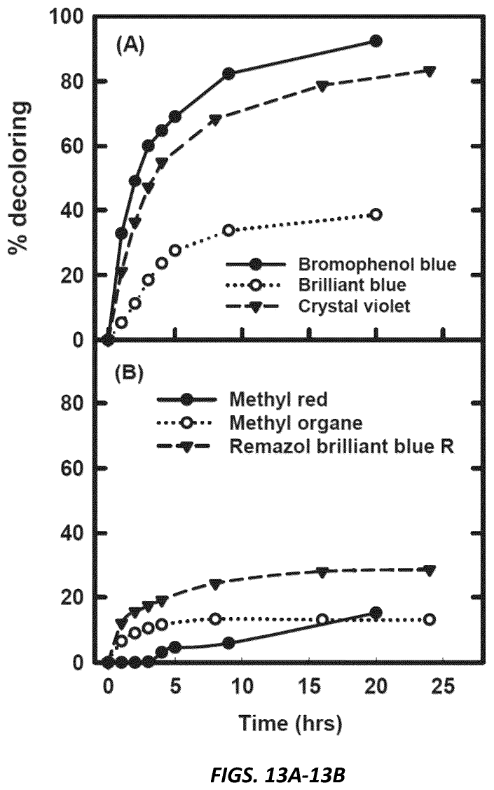

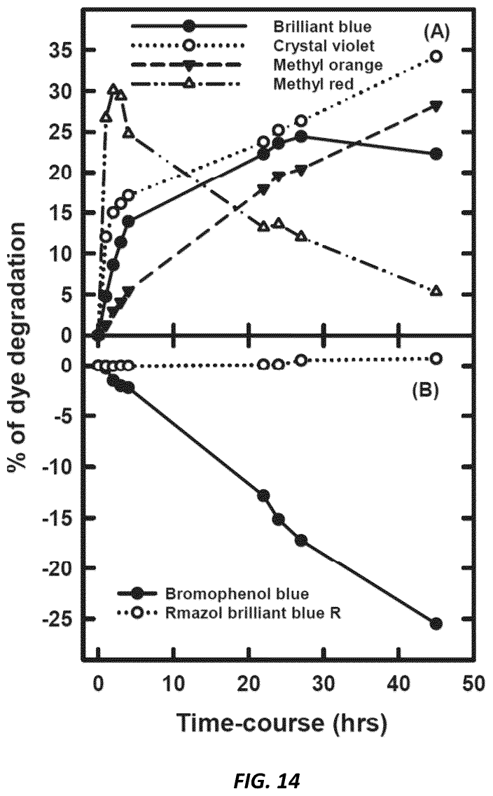

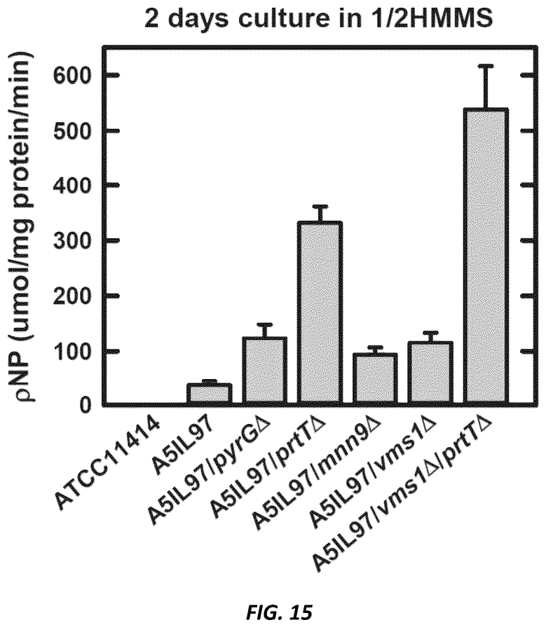

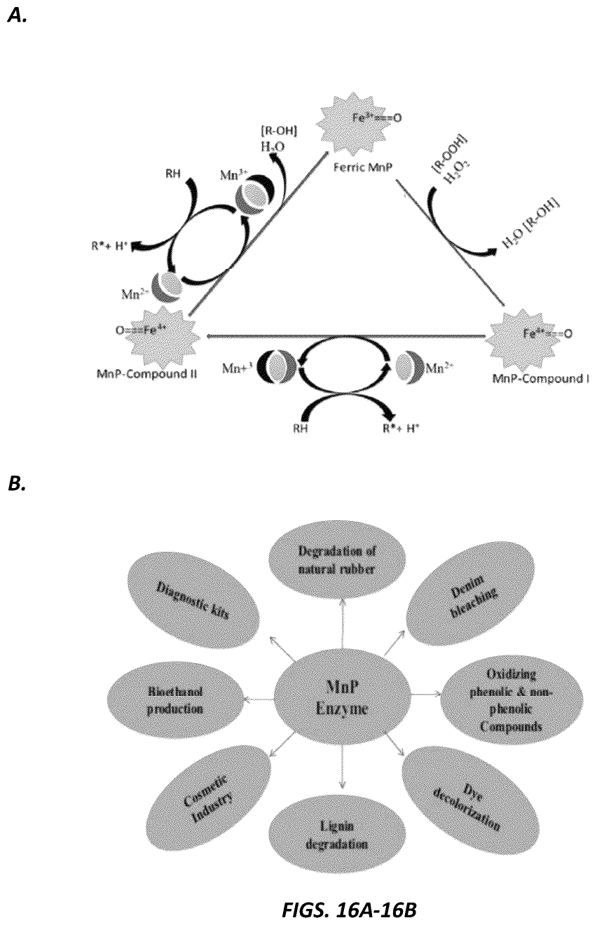

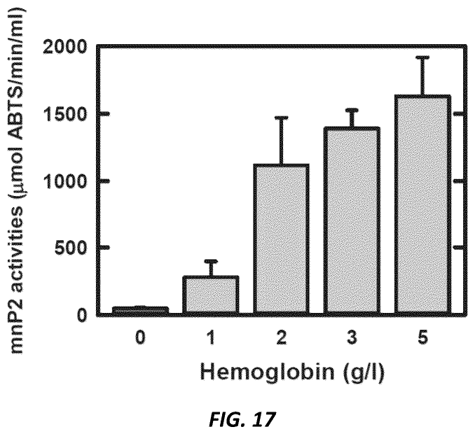

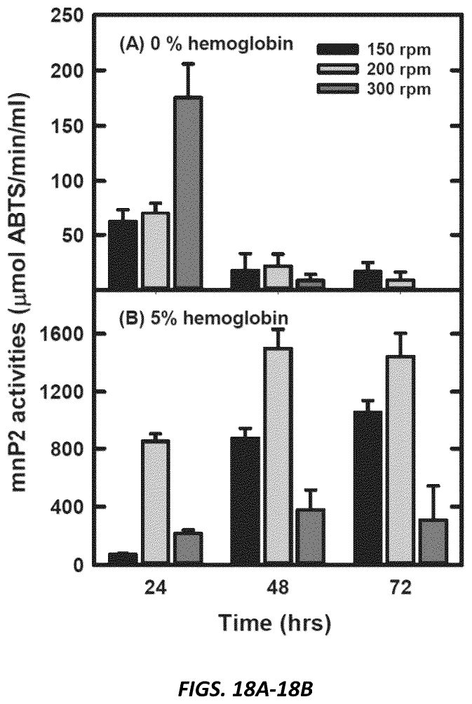

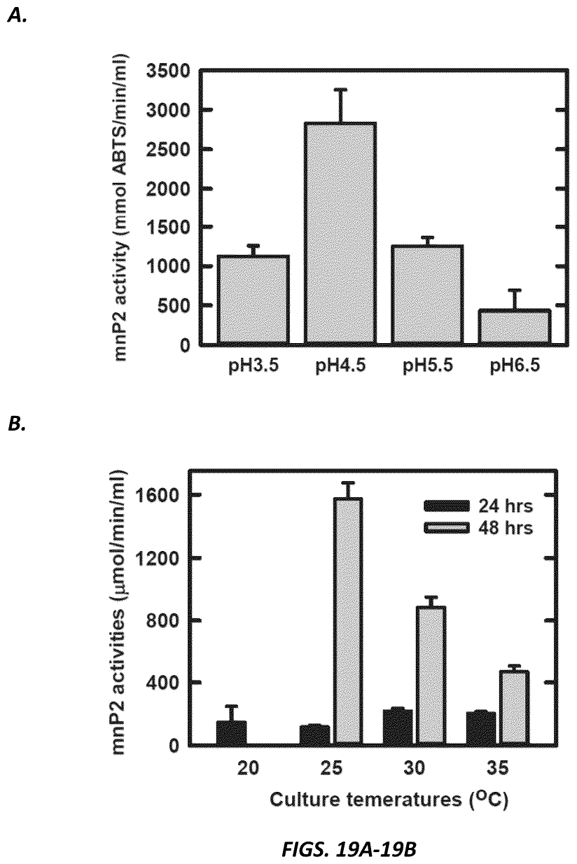

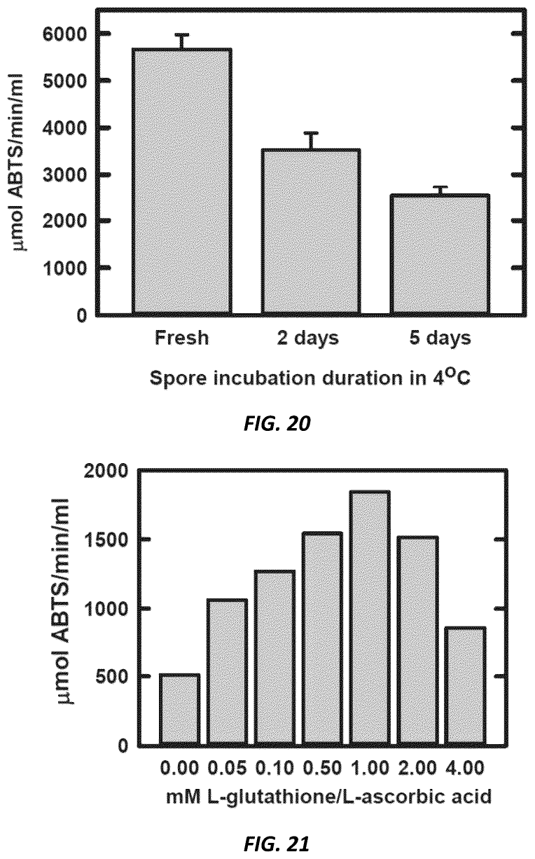

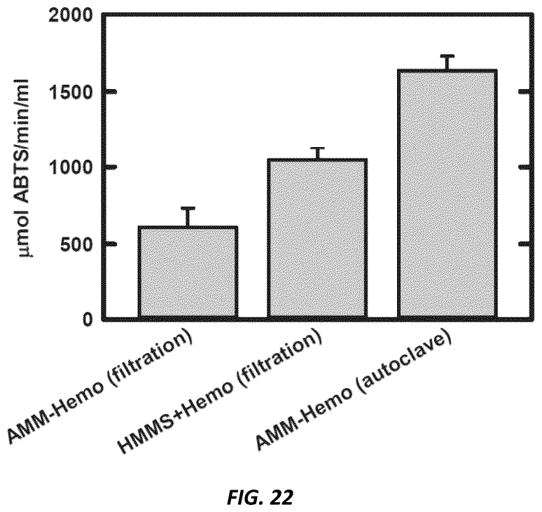

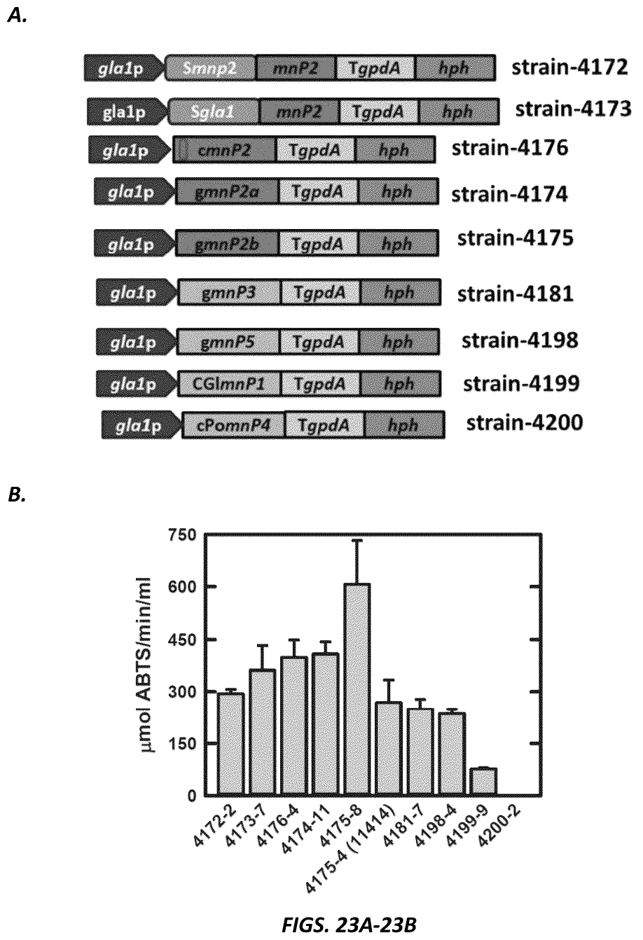

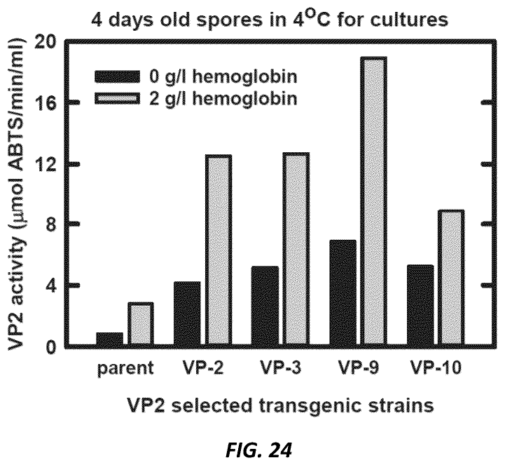

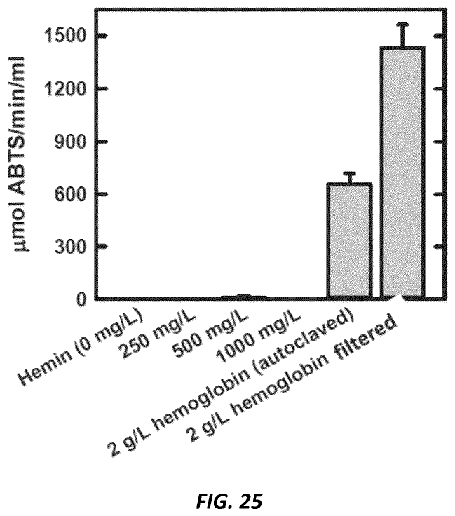

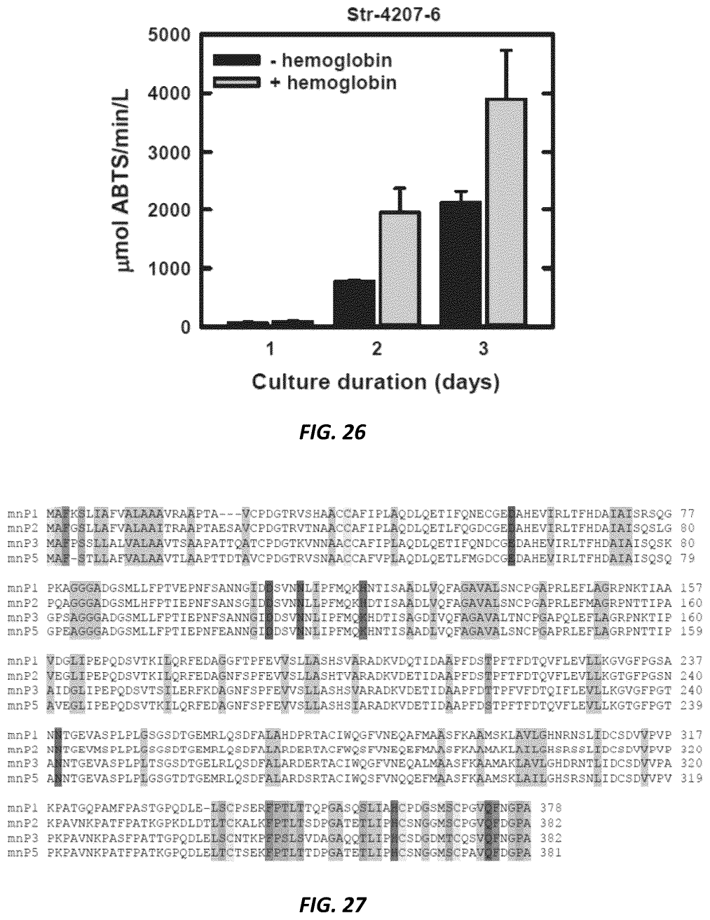

The dyes of brilliant blue, bromophenol blue, crystal violet, methyl orange, methyl red, and Remazol brilliant blue R are phenolic compounds. The culture supernatants from st-4175-8 (mnP) and 4207-6 (Lac26) were used for decoloring the selected dyes. The results in FIG. 13 show that mnP can effectively decolor the bromophenol blue, brilliant blue, and crystal violet to 93.2, 38.6, and 83.4%, respectively. The lac26 exhibits its activities in decoloring brilliant blue (34.4%), crystal violet (34.2%), and methyl orange (28.3%) with less efficiency and no activities to bromophenol blue and brilliant blue (FIG. 14). Therefore, the combined productions of selected ligninolytic enzymes will enhance lignin degradation and conversion.

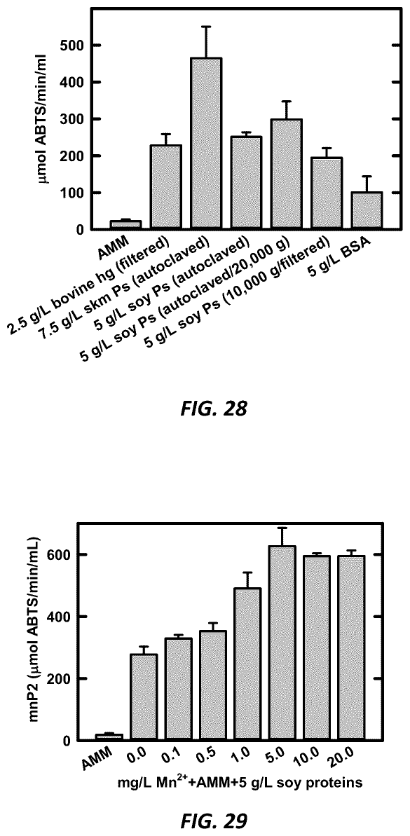

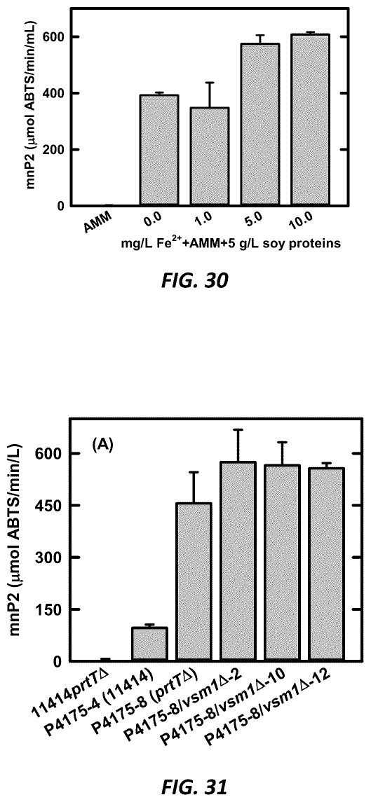

Proteins, such as bovine hemoglobin, soy protein, skim milk protein, and bovine serum albumin augment the mnP production (FIG. 28). For the mnP production, the bovine hemoglobin is the most effective and bovine serum albumin is the least effective. Addition of manganese also resulted in an increase in mnP production (FIG. 29). The results demonstrate show that additional 5 mg/L of manganese into the cultures led to the highest mnP production. Addition of additional ions were also demonstrated to increase manganese peroxidase (mnP) production. (FIGS. 30 and 31).

### BIBLIOGRAPHY

Abyzov, A., Urban, A. E., Snyder, M., Gerstein, M., 2011. CNVnator: an approach to discover, genotype, and characterize typical and atypical CNVs from family and population genome sequencing. Genome Res. 21, 974-984.
Ahmad, M., Taylor, C. R., Pink, D., Burton, K., Eastwood, D., Bending, G. D., Bugg, T. D., 2010. Development of novel assays for lignin degradation: comparative analysis of bacterial and fungal lignin degraders. Molecular biosystems. 6, 815-821.
Barton, N., Horbal, L., Starck, S., Kohlstedt, M., Luzhetskyy, A., Wittmann, C., 2018. Enabling the valorization of guaiacol-based lignin: Integrated chemical and biochemical production of cis, cis-muconic acid using metabolically engineered Amycolatopsis sp ATCC 39116. Metabolic engineering. 45, 200-210.
Bennett, J. W., Lasure, L. L., 1991. Growth media. In: Bennett, J. W. a. L., L. L., (Ed.), More Gene Manipulations in Fungi. Academic Press, San Diego, pp. 441-458.
Camarero, S., Ruiz-Dueñas, F. J., Sarkar, S., Martinez, M. J., Martinez, A. T., 2000. The cloning of a new peroxidase found in lignocellulose cultures of Pleurotus eryngii and sequence comparison with other fungal peroxidases. FEMS microbiology letters. 191, 37-43.
Chen, L., Zhang, X., Zhang, M., Zhu, Y., Zhuo, R., 2022. Removal of heavy-metal pollutants by white rot fungi: A mini review. Journal of Cleaner Production. 131681.
Chen, X., Kuhn, E., Jennings, E. W., Nelson, R., Tao, L., Zhang, M., Tucker, M. P., 2016. DMR (deacetylation and mechanical refining) processing of corn stover achieves high monomeric sugar concentrations (230 g L-1) during enzymatic hydrolysis and high ethanol concentrations (>10% v/v) during fermentation without hydrolysate purification or concentration. Energy Environ. Sci. 9, 1237-1245.
Clough, R. C., Pappu, K., Thompson, K., Beifuss, K., Lane, J., Delaney, D. E., Harkey, R., Drees, C., Howard, J. A., Hood, E. E., 2006. Manganese peroxidase from the white-rot fungus Phanerochaete chrysosporium is enzymatically active and accumulates to high levels in transgenic maize seed. Plant biotechnology journal. 4, 53-62.
Conesa, A., van den Hondel, C. A., Punt, P. J., 2000. Studies on the production of fungal peroxidases in Aspergillus niger. Applied and environmental microbiology. 66, 3016-3023. Covert, S. F., Bolduc, J., Cullen, D., 1992. Genomic organization of a cellulase gene family in Phanerochaete chrysosporium. Current genetics. 22, 407-413.
Glenn, J. K., Morgan, M. A., Mayfield, M. B., Kuwahara, M., Gold, M. H., 1983. An extracellular H2O2-requiring enzyme preparation involved in lignin biodegradation by the white rot basidiomycete Phanerochaete chrysosporium. Biochemical and biophysical research communications. 114, 1077-1083.
Hatakka, A., 1994. Lignin-modifying enzymes from selected white-rot fungi: production and role from in lignin degradation. FEMS microbiology reviews. 13, 125-135.
Jiang, B., Zhang, R., Feng, D., Wang, F., Liu, K., Jiang, Y., Niu, K., Yuan, Q., Wang, M., Wang, H., 2016. A Tet-on and Cre-lox P based genetic engineering system for convenient recycling of selection markers in Penicillium oxalicum. Front. Microbiol. 7, 485.
Kim, Y.-M., Nowack, S., Olsen, M. T., Becraft, E. D., Wood, J. M., Thiel, V., Klapper, I., Kühl, M., Fredrickson, J. K., Bryant, D. A., 2015. Diel metabolomics analysis of a hot spring chlorophototrophic microbial mat leads to new hypotheses of community member metabolisms. Front. Microbiol. 6, 209.
Kirk, T. K., Farrell, R. L., 1987. Enzymatic “combustion”: the microbial degradation of lignin. Annual Reviews in Microbiology. 41, 465-501.
Klonowska, A., Gaudin, C., Fournel, A., Asso, M., Le Petit, J., Giorgi, M., Tron, T., 2002. Characterization of a low redox potential laccase from the basidiomycete C30. European Journal of Biochemistry. 269, 6119-6125.
Kristensen, J. B., Felby, C., Jørgensen, H., 2009. Yield-determining factors in high-solids enzymatic hydrolysis of lignocellulose. Biotechnology for biofuels. 2, 1-10.
Lau, M. W., Dale, B. E., 2009. Cellulosic ethanol production from AFEX-treated corn stover using Saccharomyces cerevisiae 424A (LNH-ST). Proceedings of the National Academy of Sciences. 106, 1368-1373.
Lau, M. W., Dale, B. E., Balan, V., 2008. Ethanolic fermentation of hydrolysates from ammonia fiber expansion (AFEX) treated corn stover and distillers grain without detoxification and external nutrient supplementation. Biotechnology and bioengineering. 99, 529-539.
Lau, M. W., Gunawan, C., Balan, V., Dale, B. E., 2010. Comparing the fermentation performance of Escherichia coli KO11, Saccharomyces cerevisiae 424A (LNH-ST) and Zymomonas mobilis AX101 for cellulosic ethanol production. Biotechnology for biofuels. 3, 1-10.
Li, H., 2013. Aligning sequence reads, clone sequences and assembly contigs with BWA-MEM. arXiv preprint arXiv: 1303.3997.
Li, H., Handsaker, B., Wysoker, A., Fennell, T., Ruan, J., Homer, N., Marth, G., Abecasis, G., Durbin, R., 2009. The sequence alignment/map format and SAMtools. Bioinformatics. 25, 2078-2079.
Nakayasu, E. S., Nicora, C. D., Sims, A. C., Burnum-Johnson, K. E., Kim, Y.-M., Kyle, J. E., Matzke, M. M., Shukla, A. K., Chu, R. K., Schepmoes, A. A., 2016. MPLEx: a robust and universal protocol for single-sample integrative proteomic, metabolomic, and lipidomic analyses. MSystems. 1, e00043-16.
Pointing, S., 2001. Feasibility of bioremediation by white-rot fungi. Applied microbiology and biotechnology. 57, 20-33.
Pomraning, K. R., Dai, Z., Munoz, N., Kim, Y. M., Gao, Y., Deng, S., Kim, J., Hofstad, B. A., Swita, M. S., Lemmon, T., Collett, J. R., Panisko, E. A., Webb-Robertson, B. M., Zucker, J. D., Nicora, C. D., De Paoli, H., Baker, S. E., Burnum-Johnson, K. E., Hillson, N. J., Magnuson, J. K., 2021. Integration of Proteomics and Metabolomics Into the Design, Build, Test, Learn Cycle to Improve 3-Hydroxypropionic Acid Production in Aspergillus pseudoterreus. Front. Bioeng. Biotechnol. 9, 603832.
Reddy, C. A., 1995. The potential for white-rot fungi in the treatment of pollutants. Current opinion in Biotechnology. 6, 320-328.
Riscaldati, E., Moresi, M., Petruccioli, M., Federici, F., 2000. Effect of pH and stirring rate on itaconate production by Aspergillus terreus. J. Biotechnol. 83, 219-230.
Schell, D. J., Farmer, J., Newman, M., McMILLAN, J. D., 2003. Dilute-sulfuric acid pretreatment of corn stover in pilot-scale reactor: investigation of yields, kinetics, and enzymatic digestibilities of solids. Applied biochemistry and biotechnology. 105, 69-85.
Tien, M., Kirk, T. K., 1983. Lignin-degrading enzyme from the hymenomycete Phanerochaete chrysosporium Burds. Science. 221, 661-663.
Vanden Wymelenberg, A., Gaskell, J., Mozuch, M., Kersten, P., Sabat, G., Martinez, D., Cullen, D., 2009. Transcriptome and secretome analyses of Phanerochaete chrysosporium reveal complex patterns of gene expression. Applied and environmental microbiology. 75, 4058-4068.
Vardon, D. R., Franden, M. A., Johnson, C. W., Karp, E. M., Guarnieri, M. T., Linger, J. G., Salm, M. J., Strathmann, T. J., Beckham, G. T., 2015. Adipic acid production from lignin. Energy & Environmental Science. 8, 617-628.
Zhou, H., Leu, S.-Y., Wu, X., Zhu, J., Gleisner, R., Yang, D., Qiu, X., Horn, E., 2014. Comparisons of high titer ethanol production and lignosulfonate properties by SPORL pretreatment of lodgepole pine at two temperatures. Rsc Advances. 4, 27030-27038.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

The specific compositions and methods described herein are representative, exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nuclease” or “a cell” includes a plurality of such nucleases or cells, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72 (b) to allow the reader to quickly ascertain the nature and gist of the technical disclosure. The Abstract is submitted with the understanding that it will not be used to interpret or limit the scope or meaning of the claims.

What is claimed:

1. A modified filamentous fungus transformed with at least two polynucleotides coding for manganese peroxidase (mnP), versatile peroxidase, laccase or lignin peroxidase (lip).

2. The fungus of claim 1, wherein the fungus is transformed with polynucleotides coding for manganese peroxidase (mnP) and laccase.

3. The fungus of claim 1, wherein the fungus is transformed with polynucleotides coding for manganese peroxidase (mnP) and lip.

4. The fungus of claim 1, wherein the fungus is transformed with polynucleotides coding for manganese peroxidase (mnP), laccase, versatile peroxidase and lip.

5. The fungus of claim 1, wherein the polynucleotide comprises at least one intron.

6. The fungus of claim 1, further comprising a disruption in expression of one or more of transcriptional factor of protease (prtT), subunit of Goli mannoyltransferase complex (mnn9), and V-SNARE binding protein (vsm1) genes.

7. The fungus of claim 6, wherein expression of prtT gene is disrupted.

8. The fungus of claim 6, wherein expression of prtT and vsm1 genes is disrupted.

9. The fungus of claim 6, wherein the fungus is transformed with the manganese peroxidase polynucleotide and the prtT gene is disrupted.

10. A method of culturing the modified fugus of claim 1, comprising culturing the fungus in culture medium comprising a protein selected from hemoglobin, soy protein, skim milk protein, serum albumin or combination thereof.

11. The method of claim 10, further comprising culturing said fungus at a pH of about 4 to about 5.

12. The method of claim 10, further comprising adding manganese to the culture medium.

13. A method to increase expression of ligninolytic enzyme from a filamentous fungus comprising transforming said fungus with at least two polynucleotides encoding manganese peroxidase (mnP), versatile peroxidase, laccase or lignin peroxidase (lip) and culturing said transformed fungus so as to express at least two of manganese peroxidase (mnP), versatile peroxidase, laccase or lignin peroxidase (lip).

14. The method of claim 13, wherein the fungus is transformed with manganese peroxidase (mnP) and laccase polynucleotides.

15. The method of claim 13, wherein the fungus is transformed with manganese peroxidase (mnP) and lip polynucleotides.

16. The method of claim 14, wherein the fungus is transformed with manganese peroxidase (mnP), laccase, versatile peroxidase and lip polynucleotides.

17. The method of claim 13, wherein the polynucleotide comprises at least one intron.

18. The method of claim 13, further comprising a disruption in expression of one or more of transcriptional factor of protease (prtT), subunit of Goli mannoyltransferase complex (mnn9), and V-SNARE binding protein (vsm1) genes.

19. The method of claim 18, wherein expression of prtT gene is disrupted.

20. The method of claim 18, wherein expression of prtT and vsm1 genes is disrupted.

21. The method of claim 18, wherein the fungus is transformed with the manganese peroxidase polynucleotide and the prtT gene is disrupted.

22. The method of 13 comprising, further culturing said transformed fungus in culture medium comprising a protein selected from hemoglobin, soy protein, skim milk protein, serum albumin or combination thereof.

23. The method of claim 13, further comprising culturing said fungus at a pH of about 4 to about 5.

24. The method of claim 13, further comprising adding manganese to the culture medium.

25. The method of claim 13, further comprising harvesting one or more of manganese peroxidase (mnP), versatile peroxidase, laccase or lignin peroxidase (lip).

26. A method to degrade lignin comprising contacting said lignin with the fungus of claim 1 or one or more enzymes harvested therefrom.
